(19) United States
(12) Patent Application Publication
(10) Pub. No.: US 2017/0206313 A1
(43) Pub. Date: Jul. 20, 2017

(54) Using Flow Space Alignment to Distinguish Duplicate Reads

(71) Applicant: LIFE TECHNOLOGIES CORPORATION, Carlsbad, CA (US)

(72) Inventor: Brian Reed, Woodbridge, CT (US)

(21) Appl. No.: 15/359,773

(22) Filed: Nov. 23, 2016

Related U.S. Application Data
(62) Division of application No. 14/154,240, filed on Jan. 14, 2014, now abandoned.
(60) Provisional application No. 61/752,213, filed on Jan. 14, 2013.

(51) Int. Cl. G06F 19/22; C12Q 1/68

(52) U.S. Cl. CPC G06F 19/22; C12Q 1/6874

(57) ABSTRACT

Systems and method for identifying duplicate reads can receive first and second reads, determine if the first and second reads have a same start and end position, determine a binary flow difference, and identify the second read as a duplicate of the first read when the binary flow difference exceeds a threshold.

### CROSS-REFERENCE TO RELATED APPLICATIONS

This application is a Division of U.S. application Ser. No. 14/154,240, filed Jan. 14, 2014, which claims priority to U.S. application No. 61/752,213, filed Jan. 14, 2013, which disclosures are herein incorporated by reference in their entirety.

### SEQUENCE LISTING

The instant application contains a Sequence Listing which has been submitted electronically in ASCII format and is hereby incorporated by reference in its entirety. Said ASCII copy, created on Feb. 21, 2014, is named LT00762_SL.txt and is 1,213 bytes in size.

### FIELD

The present disclosure generally relates to the field of nucleic acid sequencing including systems and methods for distinguishing duplicate reads using flow space alignments.

### INTRODUCTION

Upon completion of the Human Genome Project, one focus of the sequencing industry has shifted to finding higher throughput and/or lower cost nucleic acid sequencing technologies, sometimes referred to as “next generation” sequencing (NGS) technologies. In making sequencing higher throughput and/or less expensive, the goal is to make the technology more accessible. These goals can be reached through the use of sequencing platforms and methods that provide sample preparation for samples of significant complexity, sequencing larger numbers of samples in parallel (for example through use of barcodes and multiplex analysis), and/or processing high volumes of information efficiently and completing the analysis in a timely manner. Various methods, such as, for example, sequencing by synthesis, sequencing by hybridization, and sequencing by ligation are evolving to meet these challenges.

Ultra-high throughput nucleic acid sequencing systems incorporating NGS technologies typically produce a large number of short sequence reads. Sequence processing methods should desirably assemble and/or map a large number of reads quickly and efficiently, such as to minimize use of computational resources. For example, data arising from sequencing of a mammalian genome can result in tens or hundreds of millions of reads that typically need to be assembled before they can be further analyzed to determine their biological, diagnostic and/or therapeutic relevance.

Exemplary applications of NGS technologies include, but are not limited to: genomic variant detection, such as insertions/deletions, copy number variations, single nucleotide polymorphisms, etc., genomic resequencing, gene expression analysis and genomic profiling.

Duplicate reads can be generated when a nucleotide fragment is amplified and results in multiple reads of the amplification products. Many techniques rely on comparing the reads spanning a region, and identifying unique reads and eliminating duplicate reads can be a significant issue. For example, gene expression analysis may rely on accurately counting a number of expression products for a gene, but duplicate reads can skew the results.

From the foregoing it will be appreciated that a need exists for systems and methods that can detect unique reads and eliminate duplicate reads from nucleic acid sequencing data.

### DRAWINGS

For a more complete understanding of the principles disclosed herein, and the advantages thereof, reference is now made to the following descriptions taken in conjunction with the accompanying drawings, in which:

FIG. 1 is a block diagram that illustrates an exemplary computer system, in accordance with various embodiments.

FIG. 2 is a schematic diagram of an exemplary system for reconstructing a nucleic acid sequence, in accordance with various embodiments.

FIG. 3 is a schematic diagram of an exemplary genetic analysis system, in accordance with various embodiments.

FIG. 4 is a flow diagram illustrating an exemplary method of identifying duplicate reads, in accordance with various embodiments.

FIG. 5 illustrates sequence data (SEQ ID NOS 1-2, respectively, in order of appearance) from two exemplary reads having the same genomic start position and flow length, but differing by a single base change, in accordance with various embodiments.

FIG. 6 is a diagram illustrating differences in the flow information for the two exemplary reads, in accordance with various embodiments.

FIG. 7 illustrates the binary flow vectors and the binary flow difference for the two exemplary reads, in accordance with various embodiments.

FIG. 8 is a flow diagram illustrating an exemplary method of classifying reads into allele groups, in accordance with various embodiments.

It is to be understood that the figures are not necessarily drawn to scale, nor are the objects in the figures necessarily drawn to scale in relationship to one another. The figures are depictions that are intended to bring clarity and understanding to various embodiments of apparatuses, systems, and methods disclosed herein. Wherever possible, the same reference numbers will be used throughout the drawings to refer to the same or like parts. Moreover, it should be appreciated that the drawings are not intended to limit the scope of the present teachings in any way.

### DESCRIPTION OF VARIOUS EMBODIMENTS

Embodiments of systems and methods for detecting low frequency variants are described herein.

The section headings used herein are for organizational purposes only and are not to be construed as limiting the described subject matter in any way.

In this detailed description of the various embodiments, for purposes of explanation, numerous specific details are set forth to provide a thorough understanding of the embodiments disclosed. One skilled in the art will appreciate, however, that these various embodiments may be practiced with or without these specific details. In other instances, structures and devices are shown in block diagram form. Furthermore, one skilled in the art can readily appreciate that the specific sequences in which methods are presented and performed are illustrative and it is contemplated that the sequences can be varied and still remain within the spirit and scope of the various embodiments disclosed herein.

All literature and similar materials cited in this application, including but not limited to, patents, patent applications, articles, books, treatises, and internet web pages are expressly incorporated by reference in their entirety for any purpose. Unless described otherwise, all technical and scientific terms used herein have a meaning as is commonly understood by one of ordinary skill in the art to which the various embodiments described herein belongs.

It will be appreciated that there is an implied “about” prior to the temperatures, concentrations, times, number of bases, coverage, etc. discussed in the present teachings, such that slight and insubstantial deviations are within the scope of the present teachings. In this application, the use of the singular includes the plural unless specifically stated otherwise. Also, the use of “comprise”, “comprises”, “comprising”, “contain”, “contains”, “containing”, “include”, “includes”, and “including” are not intended to be limiting. It is to be understood that both the foregoing general description and the following detailed description are exemplary and explanatory only and are not restrictive of the present teachings.

As used herein, “a” or “an” also may refer to “at least one” or “one or more.” Also, the use of “or” is inclusive, such that the phrase “A or B” is true when “A” is true, “B” is true, or both “A” and “B” are true.

Further, unless otherwise required by context, singular terms shall include pluralities and plural terms shall include the singular. Generally, nomenclatures utilized in connection with, and techniques of, cell and tissue culture, molecular biology, and protein and oligo- or polynucleotide chemistry and hybridization described herein are those well known and commonly used in the art. Standard techniques are used, for example, for nucleic acid purification and preparation, chemical analysis, recombinant nucleic acid, and oligonucleotide synthesis. Enzymatic reactions and purification techniques are performed according to manufacturer's specifications or as commonly accomplished in the art or as described herein. The techniques and procedures described herein are generally performed according to conventional methods well known in the art and as described in various general and more specific references that are cited and discussed throughout the instant specification. See, e.g., Sambrook et al., Molecular Cloning: A Laboratory Manual (Third ed., Cold Spring Harbor Laboratory Press, Cold Spring Harbor, N.Y. 2000). The nomenclatures utilized in connection with, and the laboratory procedures and techniques described herein are those well known and commonly used in the art.

A “system” sets forth a set of components, real or abstract, comprising a whole where each component interacts with or is related to at least one other component within the whole.

A “biomolecule” may refer to any molecule that is produced by a biological organism, including large polymeric molecules such as proteins, polysaccharides, lipids, and nucleic acids (DNA and RNA) as well as small molecules such as primary metabolites, secondary metabolites, and other natural products.

The phrase “next generation sequencing” or NGS refers to sequencing technologies having increased throughput as compared to traditional Sanger- and capillary electrophoresis-based approaches, for example with the ability to generate hundreds of thousands of relatively small sequence reads at a time. Some examples of next generation sequencing techniques include, but are not limited to, sequencing by synthesis, sequencing by ligation, and sequencing by hybridization. More specifically, the Personal Genome Machine (PGM) of Life Technologies Corp. provides massively parallel sequencing with enhanced accuracy. The PGM System and associated workflows, protocols, chemistries, etc. are described in more detail in U.S. Patent Application Publication No. 2009/0127589 and No. 2009/0026082, the entirety of each of these applications being incorporated herein by reference.

The phrase “sequencing run” refers to any step or portion of a sequencing experiment performed to determine some information relating to at least one biomolecule (e.g., nucleic acid molecule).

The phase “base space” refers to a representation of the sequence of nucleotides. The phase “flow space” refers to a representation of the incorporation event or non-incorporation event for a particular nucleotide flow. For example, flow space can be a series of values representing a nucleotide incorporation events (such as a one, “1”) or a non-incorporation event (such as a zero, “0”) for that particular nucleotide flow. Nucleotide flows having a non-incorporation event can be referred to as empty flows, and nucleotide flows having a nucleotide incorporation event can be referred to as positive flows. It should be understood that zeros and ones are convenient representations of a non-incorporation event and a nucleotide incorporation event; however, any other symbol or designation could be used alternatively to represent and/or identify these events and non-events. In particular, when multiple nucleotides are incorporated at a given position, such as for a homopolymer stretch, the value can be proportional to the number of nucleotide incorporation events and thus the length of the homopolymer stretch.

DNA (deoxyribonucleic acid) is a chain of nucleotides consisting of 4 types of nucleotides; A (adenine), T (thymine), C (cytosine), and G (guanine), and that RNA (ribonucleic acid) is comprised of 4 types of nucleotides; A, U (uracil), G, and C. Certain pairs of nucleotides specifically bind to one another in a complementary fashion (called complementary base pairing). That is, adenine (A) pairs with thymine (T) (in the case of RNA, however, adenine (A) pairs with uracil (U)), and cytosine (C) pairs with guanine (G). When a first nucleic acid strand binds to a second nucleic acid strand made up of nucleotides that are complementary to those in the first strand, the two strands bind to form a double strand. As used herein, “nucleic acid sequencing data,” “nucleic acid sequencing information,” “nucleic acid sequence,” “genomic sequence,” “genetic sequence,” or “fragment sequence,” or “nucleic acid sequencing read” denotes any information or data that is indicative of the order of the nucleotide bases (e.g., adenine, guanine, cytosine, and thymine/uracil) in a molecule (e.g., whole genome, whole transcriptome, exome, oligonucleotide, polynucleotide, fragment, etc.) of DNA or RNA. It should be understood that the present teachings contemplate sequence information obtained using all available varieties of techniques, platforms or technologies, including, but not limited to: capillary electrophoresis, microarrays, ligation-based systems, polymerase-based systems, hybridization-based systems, direct or indirect nucleotide identification systems, pyrosequencing, ion- or pH-based detection systems, electronic signature-based systems, etc.

A “polynucleotide”, “nucleic acid”, or “oligonucleotide” refers to a linear polymer of nucleosides (including deoxyribonucleosides, ribonucleosides, or analogs thereof) joined by internucleosidic linkages. Typically, a polynucleotide comprises at least three nucleosides. Usually oligonucleotides range in size from a few monomeric units, e.g. 3-4, to several hundreds of monomeric units. Whenever a polynucleotide such as an oligonucleotide is represented by a sequence of letters, such as “ATGCCTG,” it will be understood that the nucleotides are in 5′->3′ order from left to right and that “A” denotes deoxyadenosine, “C” denotes deoxycytidine, “G” denotes deoxyguanosine, and “T” denotes thymidine, unless otherwise noted. The letters A, C, G, and T may be used to refer to the bases themselves, to nucleosides, or to nucleotides comprising the bases, as is standard in the art.

As used herein, a “somatic variation” or “somatic mutation” can refer to a variation in genetic sequence that results from a mutation that occurs in a non-germline cell. The variation can be passed on to daughter cells through mitotic division. This can result in a group of cells having a genetic difference from the rest of the cells of an organism. Additionally, as the variation does not occur in a germline cell, the mutation may not be inherited by progeny organisms.

### Computer-Implemented System

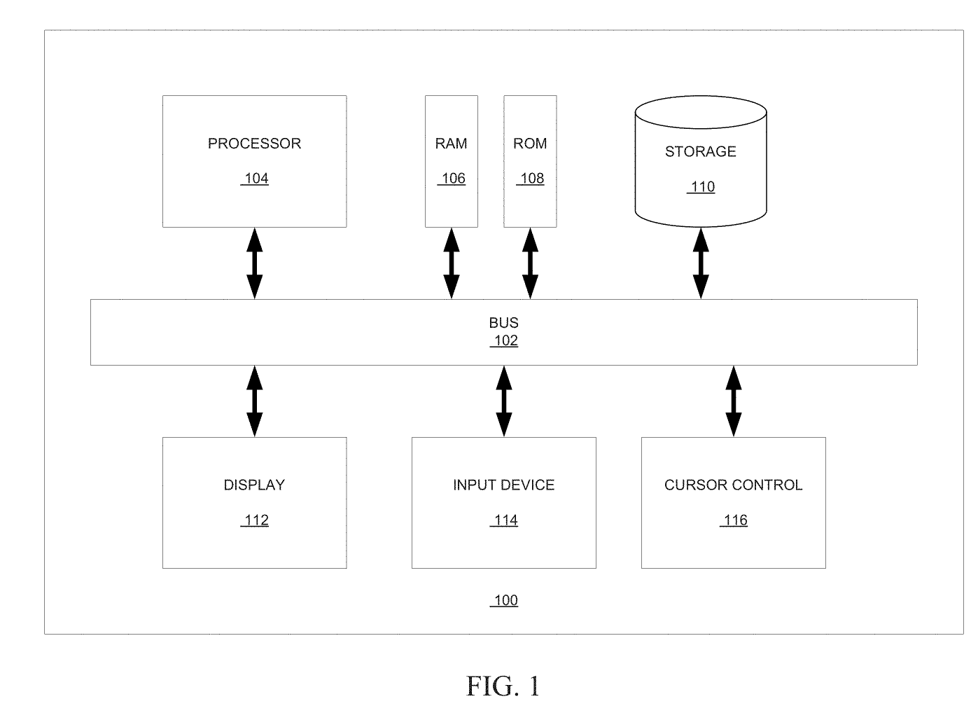

FIG. 1 is a block diagram that illustrates a computer system 100, upon which embodiments of the present teachings may be implemented. In various embodiments, computer system 100 can include a bus 102 or other communication mechanism for communicating information, and a processor 104 coupled with bus 102 for processing information. In various embodiments, computer system 100 can also include a memory 106, which can be a random access memory (RAM) or other dynamic storage device, coupled to bus 102 for determining base calls, and instructions to be executed by processor 104. Memory 106 also can be used for storing temporary variables or other intermediate information during execution of instructions to be executed by processor 104. In various embodiments, computer system 100 can further include a read only memory (ROM) 108 or other static storage device coupled to bus 102 for storing static information and instructions for processor 104. A storage device 110, such as a magnetic disk or optical disk, can be provided and coupled to bus 102 for storing information and instructions.

In various embodiments, computer system 100 can be coupled via bus 102 to a display 112, such as a cathode ray tube (CRT) or liquid crystal display (LCD), for displaying information to a computer user. An input device 114, including alphanumeric and other keys, can be coupled to bus 102 for communicating information and command selections to processor 104. Another type of user input device is a cursor control 116, such as a mouse, a trackball or cursor direction keys for communicating direction information and command selections to processor 104 and for controlling cursor movement on display 112. This input device typically has two degrees of freedom in two axes, a first axis (i.e., x) and a second axis (i.e., y), that allows the device to specify positions in a plane.

A computer system 100 can perform the present teachings. Consistent with certain implementations of the present teachings, results can be provided by computer system 100 in response to processor 104 executing one or more sequences of one or more instructions contained in memory 106. Such instructions can be read into memory 106 from another computer-readable medium, such as storage device 110. Execution of the sequences of instructions contained in memory 106 can cause processor 104 to perform the processes described herein. Alternatively hard-wired circuitry can be used in place of or in combination with software instructions to implement the present teachings. Thus implementations of the present teachings are not limited to any specific combination of hardware circuitry and software.

The term “computer-readable medium” as used herein refers to any media that participates in providing instructions to processor 104 for execution. Such a medium can take many forms, including but not limited to, non-volatile media, volatile media, and transmission media. Examples of non-volatile media can include, but are not limited to, optical or magnetic disks, such as storage device 110. Examples of volatile media can include, but are not limited to, dynamic memory, such as memory 106. Examples of transmission media can include, but are not limited to, coaxial cables, copper wire, and fiber optics, including the wires that comprise bus 102.

Common forms of non-transitory computer-readable media include, for example, a floppy disk, a flexible disk, hard disk, magnetic tape, or any other magnetic medium, a CD-ROM, any other optical medium, punch cards, paper tape, any other physical medium with patterns of holes, a RAM, PROM, and EPROM, a FLASH-EPROM, any other memory chip or cartridge, or any other tangible medium from which a computer can read.

In accordance with various embodiments, instructions configured to be executed by a processor to perform a method are stored on a computer-readable medium. The computer-readable medium can be a device that stores digital information. For example, a computer-readable medium includes a compact disc read-only memory (CD-ROM) as is known in the art for storing software. The computer-readable medium is accessed by a processor suitable for executing instructions configured to be executed.

### Nucleic Acid Sequencing Platforms

Nucleic acid sequence data can be generated using various techniques, platforms or technologies, including, but not limited to: capillary electrophoresis, microarrays, ligation-based systems, polymerase-based systems, hybridization-based systems, direct or indirect nucleotide identification systems, pyrosequencing, ion- or pH-based detection systems, electronic signature-based systems, etc.

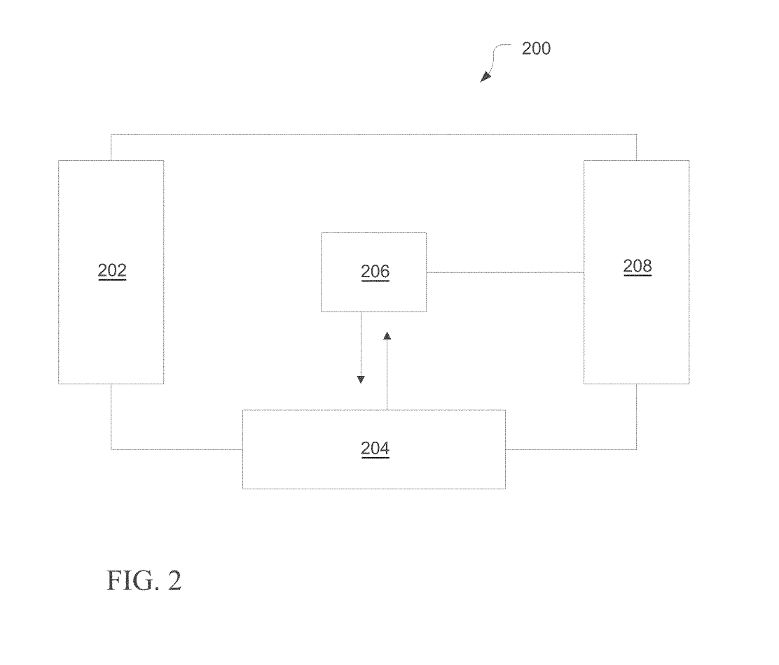

Various embodiments of nucleic acid sequencing platforms, such as a nucleic acid sequencer, can include components as displayed in the block diagram of FIG. 2. According to various embodiments, sequencing instrument 200 can include a fluidic delivery and control unit 202, a sample processing unit 204, a signal detection unit 206, and a data acquisition, analysis and control unit 208. Various embodiments of instrumentation, reagents, libraries and methods used for next generation sequencing are described in U.S. Patent Application Publication No. 2009/0127589 and No. 2009/0026082 are incorporated herein by reference. Various embodiments of instrument 200 can provide for automated sequencing that can be used to gather sequence information from a plurality of sequences in parallel, such as substantially simultaneously.

In various embodiments, the fluidics delivery and control unit 202 can include reagent delivery system. The reagent delivery system can include a reagent reservoir for the storage of various reagents. The reagents can include RNA-based primers, forward/reverse DNA primers, oligonucleotide mixtures for ligation sequencing, nucleotide mixtures for sequencing-by-synthesis, optional ECC oligonucleotide mixtures, buffers, wash reagents, blocking reagent, stripping reagents, and the like. Additionally, the reagent delivery system can include a pipetting system or a continuous flow system which connects the sample processing unit with the reagent reservoir.

In various embodiments, the sample processing unit 204 can include a sample chamber, such as flow cell, a substrate, a micro-array, a multi-well tray, or the like. The sample processing unit 204 can include multiple lanes, multiple channels, multiple wells, or other means of processing multiple sample sets substantially simultaneously. Additionally, the sample processing unit can include multiple sample chambers to enable processing of multiple runs simultaneously. In particular embodiments, the system can perform signal detection on one sample chamber while substantially simultaneously processing another sample chamber. Additionally, the sample processing unit can include an automation system for moving or manipulating the sample chamber.

In various embodiments, the signal detection unit 206 can include an imaging or detection sensor. For example, the imaging or detection sensor can include a CCD, a CMOS, an ion or chemical sensor, such as an ion sensitive layer overlying a CMOS or FET, a current or voltage detector, or the like. The signal detection unit 206 can include an excitation system to cause a probe, such as a fluorescent dye, to emit a signal. The excitation system can include an illumination source, such as arc lamp, a laser, a light emitting diode (LED), or the like. In particular embodiments, the signal detection unit 206 can include optics for the transmission of light from an illumination source to the sample or from the sample to the imaging or detection sensor. Alternatively, the signal detection unit 206 may provide for electronic or non-photon based methods for detection and consequently not include an illumination source. In various embodiments, electronic-based signal detection may occur when a detectable signal or species is produced during a sequencing reaction. For example, a signal can be produced by the interaction of a released byproduct or moiety, such as a released ion, such as a hydrogen ion, interacting with an ion or chemical sensitive layer. In other embodiments a detectable signal may arise as a result of an enzymatic cascade such as used in pyrosequencing (see, for example, U.S. Patent Application Publication No. 2009/0325145, the entirety of which being incorporated herein by reference) where pyrophosphate is generated through base incorporation by a polymerase which further reacts with ATP sulfurylase to generate ATP in the presence of adenosine 5′ phosphosulfate wherein the ATP generated may be consumed in a luciferase mediated reaction to generate a chemiluminescent signal. In another example, changes in an electrical current can be detected as a nucleic acid passes through a nanopore without the need for an illumination source.

In various embodiments, a data acquisition analysis and control unit 208 can monitor various system parameters. The system parameters can include temperature of various portions of instrument 200, such as sample processing unit or reagent reservoirs, volumes of various reagents, the status of various system subcomponents, such as a manipulator, a stepper motor, a pump, or the like, or any combination thereof.

It will be appreciated by one skilled in the art that various embodiments of instrument 200 can be used to practice variety of sequencing methods including ligation-based methods, sequencing by synthesis, single molecule methods, nanopore sequencing, and other sequencing techniques.

In various embodiments, the sequencing instrument 200 can determine the sequence of a nucleic acid, such as a polynucleotide or an oligonucleotide. The nucleic acid can include DNA or RNA, and can be single stranded, such as ssDNA and RNA, or double stranded, such as dsDNA or a RNA/cDNA pair. In various embodiments, the nucleic acid can include or be derived from a fragment library, a mate pair library, a ChIP fragment, or the like. In particular embodiments, the sequencing instrument 200 can obtain the sequence information from a single nucleic acid molecule or from a group of substantially identical nucleic acid molecules.

In various embodiments, sequencing instrument 200 can output nucleic acid sequencing read data in a variety of different output data file types/formats, including, but not limited to: *.fasta, *.csfasta, *seq.txt, *qseq.txt, *.fastq, *.sff, *prb.txt, *.sms, *srs and/or *.qv.

### System and Methods for Identifying Sequence Variation

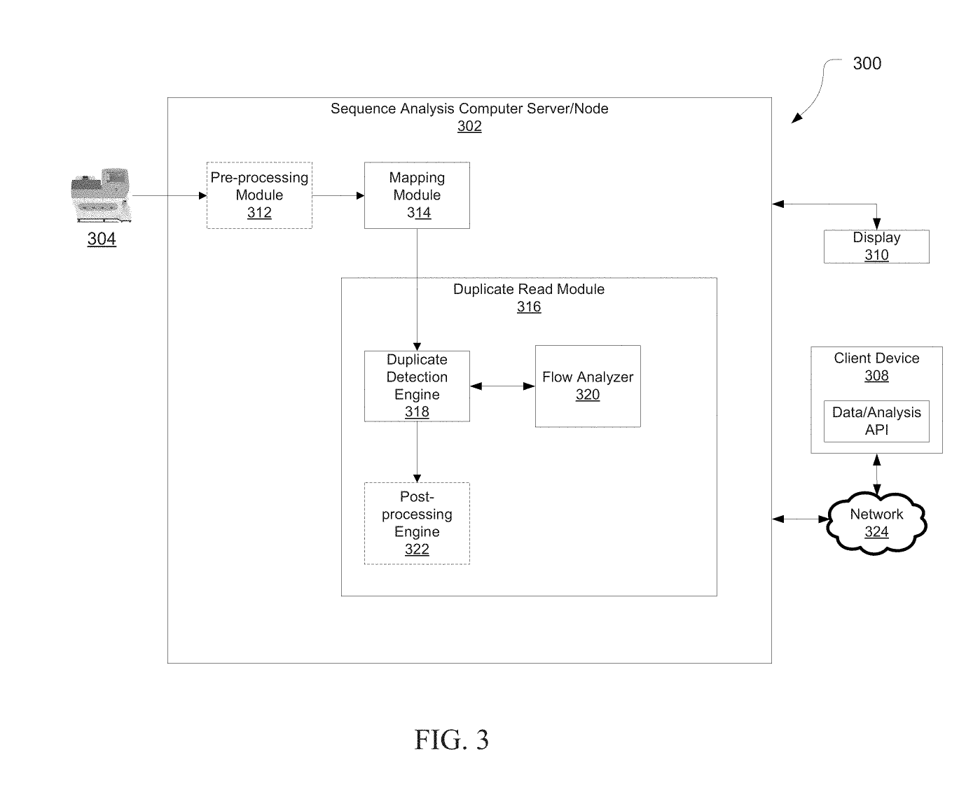

FIG. 3 is a schematic diagram of a system for identifying variants, in accordance with various embodiments.

As depicted herein, variant analysis system 300 can include a nucleic acid sequence analysis device 304 (e.g., nucleic acid sequencer, real-time/digital/quantitative PCR instrument, microarray scanner, etc.), an analytics computing server/node/device 302, and a display 310 and/or a client device terminal 308.

In various embodiments, the analytics computing sever/node/device 302 can be communicatively connected to the nucleic acid sequence analysis device 304, and client device terminal 308 via a network connection 324 that can be either a “hardwired” physical network connection (e.g., Internet, LAN, WAN, VPN, etc.) or a wireless network connection (e.g., Wi-Fi, WLAN, etc.).

In various embodiments, the analytics computing device/server/node 302 can be a workstation, mainframe computer, distributed computing node (such as, part of a “cloud computing” or distributed networking system), personal computer, mobile device, etc. In various embodiments, the nucleic acid sequence analysis device 304 can be a nucleic acid sequencer, real-time/digital/quantitative PCR instrument, microarray scanner, etc. It should be understood, however, that the nucleic acid sequence analysis device 304 can essentially be any type of instrument that can generate nucleic acid sequence data from samples obtained from an individual.

The analytics computing server/node/device 302 can be configured to host an optional pre-processing module 312, a mapping module 314, and a duplicate read module 316.

Pre-processing module 312 can be configured to receive from the nucleic acid sequence analysis device 304 and perform processing steps, such as conversion from f space to base space, color space to base space, or from flow space to base space, determining call quality values, preparing the read data for use by the mapping module 314, and the like.

The mapping module 314 can be configured to align (i.e., map) a nucleic acid sequence read to a reference sequence. Generally, the length of the sequence read is substantially less than the length of the reference sequence. In reference sequence mapping/alignment, sequence reads are assembled against an existing backbone sequence (e.g., reference sequence, etc.) to build a sequence that is similar but not necessarily identical to the backbone sequence. Once a backbone sequence is found for an organism, comparative sequencing or re-sequencing can be used to characterize the genetic diversity within the organism's species or between closely related species. In various embodiments, the reference sequence can be a whole/partial genome, whole/partial exome, etc.

In various embodiments, the sequence read and reference sequence can be represented as a sequence of nucleotide base symbols in base space. In various embodiments, the sequence read and reference sequence can be represented as one or more colors in color space. In various embodiments, the sequence read and reference sequence can be represented as nucleotide base symbols with signal or numerical quantitation components in flow space.

In various embodiments, the alignment of the sequence fragment and reference sequence can include a limited number of mismatches between the bases that comprise the sequence fragment and the bases that comprise the reference sequence. Generally, the sequence fragment can be aligned to a portion of the reference sequence in order to minimize the number of mismatches between the sequence fragment and the reference sequence.

The duplicate read module 316 can include a duplicate detection engine 318, a flow analyzer 320, and an optional post processing engine 322. In various embodiments, duplicate read module 316 can be in communications with the mapping module 314. That is, duplicate read module 316 can request and receive data and information (through, e.g., data streams, data files, text files, etc.) from mapping module 314.

The duplicate detection engine 318 can be configured to receive mapped reads from the mapping module 314, identify reads having the same start and end position, and provide reads to the flow analyzer 320. In various embodiments, a read can include a portion of a start tag, an insert, and a portion of an end tag. The tag can include a portion of an adapter sequence, a barcode, a key sequence, or the like. Generally, the tag can be a known or identifiable portion of the sequence that is not part of the insert or target sequence. In various embodiments, the start position can be a genomic coordinate where the first base of an insert is mapped to a reference sequence, while the end position can be a flow position of the last base of the insert or the first base of the end tag. The duplicate detection engine 318 can receive a binary flow difference for a pair of reads from the flow analyzer 320 and identify duplicate reads based on the binary flow difference.

Flow analyzer 320 can be configured to receive reads from the duplicate detection engine 318, determine a binary flow difference, and return the binary flow difference to the duplicate detection engine 318. The flow analyzer 320 can generate a binary flow vector for the reads having the same start and end positions. The binary flow vector can have a zero value for each empty flow and a 1 value for each positive flow in the flow series. The flow analyzer 320 can determine the binary flow difference by summing the absolute value of the difference across the binary flow vectors.

Post processing engine 322 can be configured to receive the duplicate reads identified by the duplicate detection engine 318 and perform additional processing steps, such as filtering duplicate reads, and formatting the read data for display on display 310 or use by client device 308.

Client device 308 can be a thin client or thick client computing device. In various embodiments, client terminal 308 can have a web browser (e.g., INTERNET EXPLORER™, FIREFOX™, SAFARI™, etc) that can be used to communicate information to and/or control the operation of the pre-processing module 312, mapping module 314, duplicate detection engine 318, flow analyzer 320, and post processing engine 322 using a browser to control their function. For example, the client terminal 308 can be used to configure the operating parameters (e.g., match scoring parameters, annotations parameters, filtering parameters, data security and retention parameters, etc.) of the various modules, depending on the requirements of the particular application. Similarly, client terminal 308 can also be configure to display the results of the analysis performed by the duplicate read module 316 and the nucleic acid sequencer 304.

It should be understood that the various data stores disclosed as part of system 300 can represent hardware-based storage devices (e.g., hard drive, flash memory, RAM, ROM, network attached storage, etc.) or instantiations of a database stored on a standalone or networked computing device(s).

It should also be appreciated that the various data stores and modules/engines shown as being part of the system 300 can be combined or collapsed into a single module/engine/data store, depending on the requirements of the particular application or system architecture. Moreover, in various embodiments, the system 300 can comprise additional modules, engines, components or data stores as needed by the particular application or system architecture.

In various embodiments, the system 300 can be configured to process the nucleic acid reads in color space. In various embodiments, system 300 can be configured to process the nucleic acid reads in base space. In various embodiments, system 300 can be configured to process the nucleic acid sequence reads in flow space. It should be understood, however, that the system 300 disclosed herein can process or analyze nucleic acid sequence data in any schema or format as long as the schema or format can convey the base identity and position of the nucleic acid sequence.

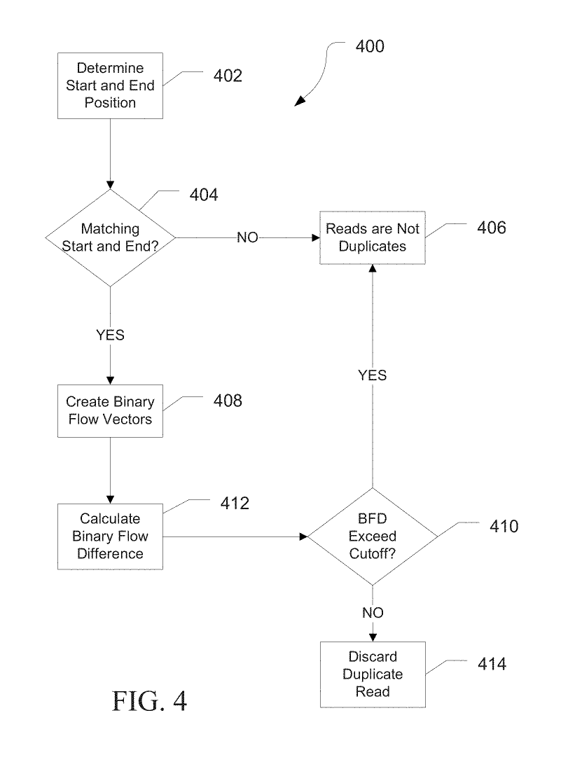

FIG. 4 is an exemplary flow diagram showing a method 400 for identifying duplicate nucleic acid sequence reads, in accordance with various embodiments.

At 402, start and end positions can be determined for a plurality of reads from a sample. The read may include portions of a start tag, an insert, and portions of an end tag. For example, the start and end tag may correspond to portions of amplification primers used to amplify the sample, or they may correspond to tags ligated to the ends of an nucleic acid from the sample, such as to provide a sequence to enable priming of the sequencing reaction. In exemplary embodiments, the start position can be a genomic coordinate corresponding to a location in a reference genome to which a read maps. For example, the genomic coordinate can indicate the mapped position of the first base of an insert from the read. In exemplary embodiments, the end position can be a flow space position of the end of the insert or the beginning of an end tag. In various embodiments, the boundaries of the insert can be identified, and the insert can be mapped to a reference genome to identify the start and end portions of the reads.

At 404, it can be determined if the start and end portions of a pair of reads match. In particular embodiments, a list of reads can be sorted by the genomic coordinate of the start position. Reads having the same genomic start position can be pooled based on the end position to identify reads mapping to the same portion of the reference sequence that have the same flow length for the insert. In various embodiments, due to the presence of empty flows in a flow sequence, the flow length of a read may be different even for reads having the same length of insert.

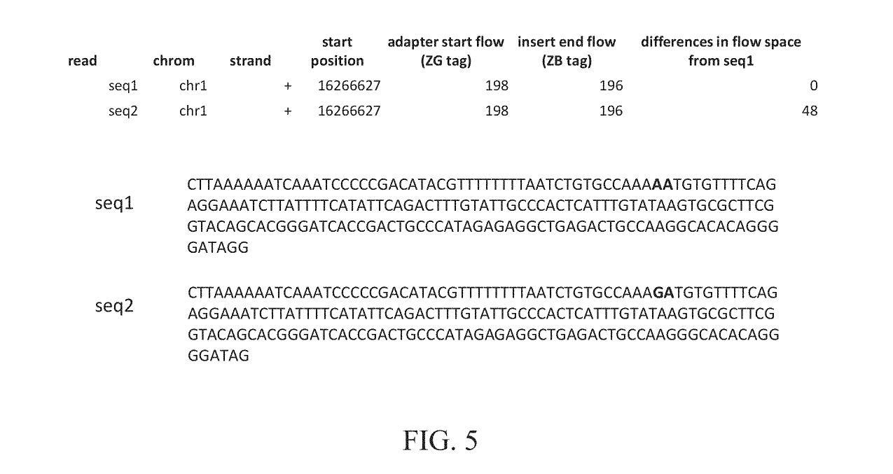

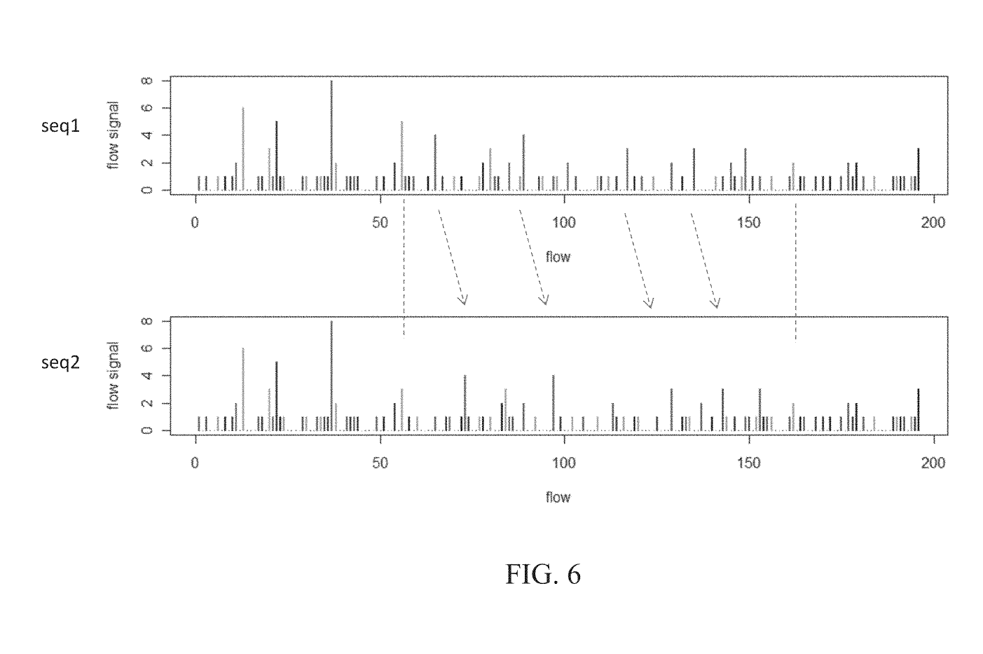

By way of an example, FIG. 5 illustrates two exemplary reads (seq1 and seq2) having the same start position and insert flow length. Seq2 differs from seq1 by a single A->G substitution, identified in bold. FIG. 6 illustrates the flow data for seq1 and seq2. As illustrates by the arrows, the changes in the flow data caused by the A->G substitution extend for approximately 100 flows, in this particular example.

Returning to FIG. 4, when the genomic start position and the insert flow length are not the same, then the reads are not duplicates, as indicated at 406. Alternatively, at 408, when reads have the same genomic start position and insert flow length, binary flow vectors can be created for the reads. A binary flow vector can be a simplified version of a flow space representation. Specifically, empty flows can be designated with a zero value and positive flows can be designated with a one value.

At 410, a binary flow difference can be calculated for reads having common start and end positions. Specifically, the absolute value of the difference between the binary flow vectors can be summed over the flow length of the insert. In essence, the binary flow difference provides a count of the nucleotide flows when one read has an empty flow and the other read has a positive flow.

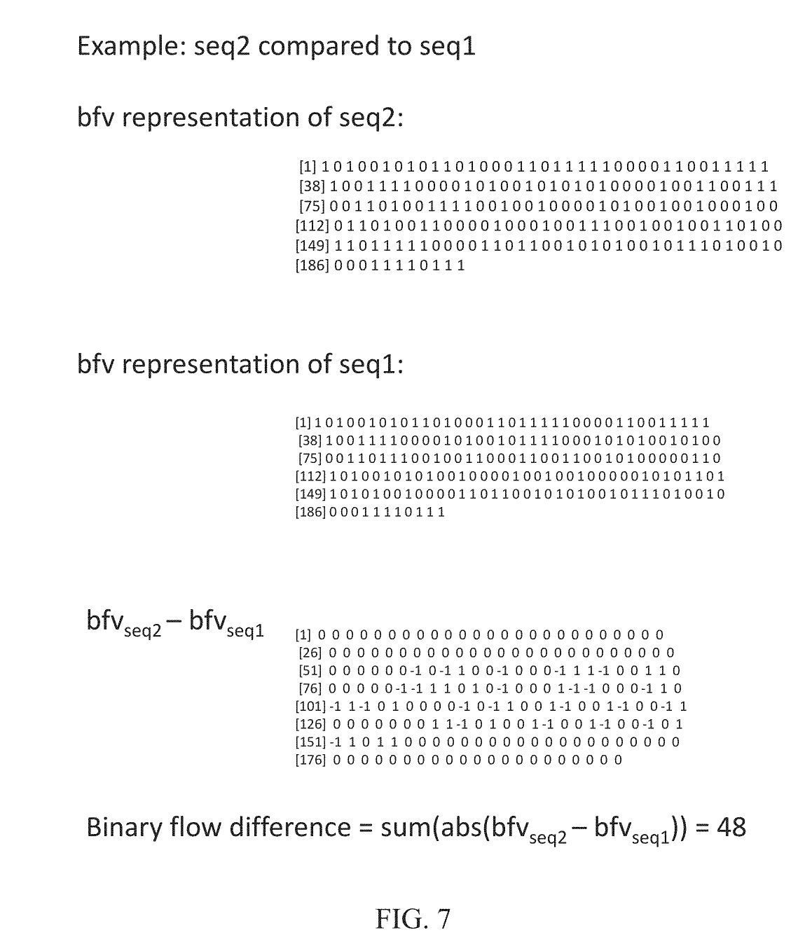

FIG. 7 illustrates the exemplary binary flow vectors of seq1 and seq2, and the exemplary binary flow vector difference between seq1 and seq2.

Returning to FIG. 4, at 412, it can be determined if the binary flow difference exceeds a cutoff. Large binary flow differences are indicative of a reliable sequence difference between the reads, rather than a sequence difference due to a sequencing error. When the binary flow difference exceeds a threshold, the reads are not duplicates, as illustrated at 406. Alternatively, when the binary flow difference is below the threshold, the reads may be duplicate reads, and all but one of the reads may be discarded.

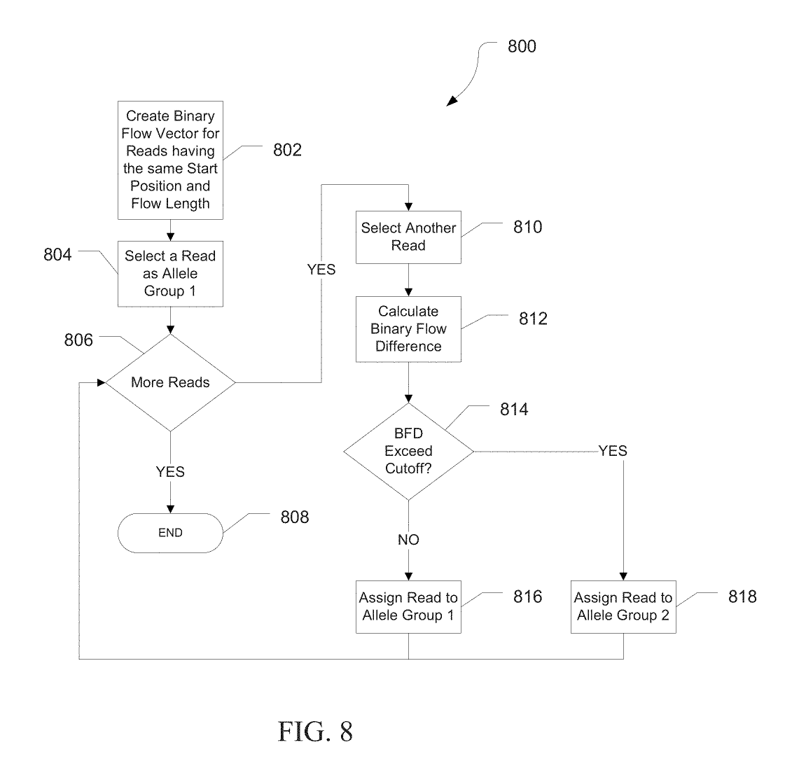

FIG. 8 is an exemplary flow diagram showing a method 800 for classifying nucleic acid sequence reads into allele groups, in accordance with various embodiments.

At 802, binary flow vectors can be created for reads having the same genomic start position and insert flow length. At 804, one of the reads can be selected as a representative of allele group 1. At 806, a determination can be made if there are any more reads having the same genomic start position and insert flow length. When there are no more reads, the method can end at 808.

Alternatively, at 810, a read to be classified can be selected. At 812, a binary flow difference can be calculated between the read to be classified and the representative read.

At 814, it can be determined if the binary flow difference exceeds a cutoff. When the binary flow difference is below the threshold, the read can be classified as an allele group 1 read, as illustrated at 816. Alternatively, when the binary flow difference exceeds the threshold, the reads can be classified as an allele group 2 read, as illustrated at 818.

In various embodiments, reads in allele group 2 can be further classified following a similar procedure to determine if any reads belong to additional allele groups.

In various embodiments, the methods of the present teachings may be implemented in a software program and applications written in conventional programming languages such as C, C++, etc.

While the present teachings are described in conjunction with various embodiments, it is not intended that the present teachings be limited to such embodiments. On the contrary, the present teachings encompass various alternatives, modifications, and equivalents, as will be appreciated by those of skill in the art.

Further, in describing various embodiments, the specification may have presented a method and/or process as a particular sequence of steps. However, to the extent that the method or process does not rely on the particular order of steps set forth herein, the method or process should not be limited to the particular sequence of steps described. As one of ordinary skill in the art would appreciate, other sequences of steps may be possible. Therefore, the particular order of the steps set forth in the specification should not be construed as limitations on the claims. In addition, the claims directed to the method and/or process should not be limited to the performance of their steps in the order written, and one skilled in the art can readily appreciate that the sequences may be varied and still remain within the spirit and scope of the various embodiments.

The embodiments described herein, can be practiced with other computer system configurations including hand-held devices, microprocessor systems, microprocessor-based or programmable consumer electronics, minicomputers, mainframe computers and the like. The embodiments can also be practiced in distributing computing environments where tasks are performed by remote processing devices that are linked through a network.

It should also be understood that the embodiments described herein can employ various computer-implemented operations involving data stored in computer systems. These operations are those requiring physical manipulation of physical quantities. Usually, though not necessarily, these quantities take the form of electrical or magnetic signals capable of being stored, transferred, combined, compared, and otherwise manipulated. Further, the manipulations performed are often referred to in terms, such as producing, identifying, determining, or comparing.

Any of the operations that form part of the embodiments described herein are useful machine operations. The embodiments, described herein, also relate to a device or an apparatus for performing these operations. The systems and methods described herein can be specially constructed for the required purposes or it may be a general purpose computer selectively activated or configured by a computer program stored in the computer. In particular, various general purpose machines may be used with computer programs written in accordance with the teachings herein, or it may be more convenient to construct a more specialized apparatus to perform the required operations.

Certain embodiments can also be embodied as computer readable code on a computer readable medium. The computer readable medium is any data storage device that can store data, which can thereafter be read by a computer system. Examples of the computer readable medium include hard drives, network attached storage (NAS), read-only memory, random-access memory, CD-ROMs, CD-Rs, CD-RWs, magnetic tapes, and other optical and non-optical data storage devices. The computer readable medium can also be distributed over a network coupled computer systems so that the computer readable code is stored and executed in a distributed fashion.

1.-20. (canceled)

21. A method of identifying a duplicate reads in a plurality of reads produced by a nucleic acid sequencing device, comprising:
mapping the reads to a reference genome to determine genomic start positions for the reads using a processor;
identifying an end of an insert or a beginning of an end tag in the reads to determine flow end positions for the reads;
identifying a pair of reads having the same genomic start position and flow end position;
generating binary flow vectors for the pair of reads;
calculating a binary flow difference for the pair of reads; and
identifying the pair of reads as duplicate reads when the binary flow difference is below a threshold.

22. The method of claim 21, wherein the plurality of reads are related to a plurality of template polynucleotide strands disposed in a sample processing unit of the nucleic acid sequencing device, the template polynucleotide strands having been exposed to a series of flows of nucleotide species.

23. The method of claim 21, wherein the binary flow vector has a value of 0 for an empty flow and a value of 1 for a positive flow corresponding to an incorporation event.

24. The method of claim 21, wherein the binary flow difference is a sum of absolute values of differences between the binary flow vectors across all flows.

25. The method of claim 21, wherein the reads can contain sequence information from a start tag, an insert, and an end tag.

26. The method of claim 25, further comprising identifying an end of the start tag or a beginning of the insert.

27. A system for identifying duplicate reads in a plurality of reads produced by a nucleic acid sequencing device, comprising a processor configured to:
receive the plurality of reads from the nucleic acid sequencing device,
map the reads to a reference genome to determine genomic start positions and flow end positions,
identify reads having the same genomic start position and flow end position,
generate binary flow vectors for the reads having the same genomic start position and flow end position,
determine a binary flow difference between the binary flow vectors, and
identify reads as duplicate reads when the binary flow difference is below a threshold.

28. The system of claim 27, wherein the nucleic acid sequencing device is configured to expose a plurality of template polynucleotide strands disposed in a sample processing unit of the nucleic acid sequencing device to a series of flows of nucleotide species and to obtain the plurality of reads related to the plurality of template polynucleotide strands by measuring signals representing incorporation of the nucleotide species.

29. The system of claim 27, wherein the binary flow vector has a value of 0 for an empty flow and a value of 1 for a positive flow corresponding to an incorporation event.

30. The system of claim 27, wherein the binary flow difference is a sum of absolute values of differences between the binary flow vectors across all flows.

31. The system of claim 27, wherein the reads can contain sequence information from a start tag, an insert, and an end tag.

32. The system of claim 31, wherein the processor is further configured to identify an end of the start tag or a beginning of the insert.

33. A method of identifying a classifying reads in a plurality of reads produced by a nucleic acid sequencing device, comprising:
mapping the plurality of reads to a reference genome to determine genomic start positions for the reads;
identifying an end of an insert or a beginning of an end tag in the reads to determine flow end positions for the reads;
identifying a set of reads having the same genomic start position and flow end position;
generating binary flow vectors for the reads of the set;
calculating binary flow differences between the reads of the set and a first read of the set; and
classifying the reads of the set into first and second allele groups based on the binary flow differences.

34. The method of claim 33, wherein the plurality of reads are related to a plurality of template polynucleotide strands disposed in a sample processing unit of the nucleic acid sequencing device, the template polynucleotide strands having been exposed to a series of flows of nucleotide species.

35. The method of claim 33, wherein the binary flow vector has a value of 0 for an empty flow and a value of 1 for a positive flow corresponding to an incorporation event.

36. The method of claim 33, wherein the binary flow difference is a sum of absolute values of differences between the binary flow vectors across all flows.

37. The method of claim 33, wherein the classifying the reads of the set includes classifying a particular read of the set into the second allele group when the binary flow difference exceeds a threshold.

38. The method of claim 33, wherein the classifying the reads of the set includes classifying a particular read of the set into the first allele group when the binary flow difference does not exceeds a threshold.

39. The method of claim 33, wherein the reads can contain sequence information from a start tag, an insert, and an end tag.

40. The method of claim 39, further comprising identifying an end of the start tag or a beginning of the insert.


SEQUENCE LISTING (2 sequences)

SEQ ID NO: 1 - DNA, 189 bp, Artificial Sequence
  Description of Artificial Sequence Synthetic polynucleotide
cttaaaaaatcaaatcccccgacatacgttttttttaatctgtgccaaaaatgtgttttcagaggaaatcttattttcatattcagactttgtattgcccactcatttgtataagtgcgcttcggtacagcacgggatcaccgactgcccatagagaggctgagactgccaaggcacacaggggatagg

SEQ ID NO: 2 - DNA, 189 bp, Artificial Sequence
  Description of Artificial Sequence Synthetic polynucleotide
cttaaaaaatcaaatcccccgacatacgttttttttaatctgtgccaaagatgtgttttcagaggaaatcttattttcatattcagactttgtattgcccactcatttgtataagtgcgcttcggtacagcacgggatcaccgactgcccatagagaggctgagactgccaagggcacacaggggatag